(19) United States
(12) Patent Application Publication
(10) Pub. No.: US 2017/0367764 A1
(43) Pub. Date: Dec. 28, 2017

(54) COMPUTER ASSISTED IMPLANT PLACEMENT

(71) Applicant: THINK SURGICAL, INC., Fremont, CA (US)

(72) Inventors: Joel ZUHARS, Fremont, CA (US); Daniel Patrick BONNY, Fremont, CA (US)

(73) Assignee: THINK SURGICAL, INC., Fremont, CA (US)

(21) Appl. No.: 15/542,707

(22) PCT Filed: Jan. 15, 2016
(86) PCT No.: PCT/US16/13533

§ 371 Date: Jul. 11, 2017

Related U.S. Application Data
(60) Provisional application No. 62/104,657, filed on Jan. 16, 2015.

(51) Int. Cl. A61B 34/10; A61F 2/46; A61F 2/36; A61F 2/30

(52) U.S. Cl. CPC A61B 34/10; A61F 2/3609; A61F 2/4607; A61F 2/4609; A61B 2034/101; A61F 2002/30604; A61F 2002/3623; A61F 2002/4633; A61F 2002/30253

(57) ABSTRACT

A method for implantation of non-spherical, asymmetric implants is provided that includes devising a pre-surgical plan with pre-operative planning software operating on a computer to define at least one of shape, orientation, type, size, geometry, or placement of the non-spherical, asymmetric implant in an operative bone of a subject. A computer assisted surgical device is used to place the non-spherical, asymmetric implant. The implant is positioned within the bone by the computer assisted surgical device in accordance with pre-surgical plan. A non-spherical, asymmetric implant for insertion in a bone formed of separate stem, neck, and head portions and suitable for implantation by the method is also disclosed.

### RELATED APPLICATIONS

This application claims priority benefit of U.S. Provisional Application Ser. No. 62/104,657 filed Jan. 16, 2015; the contents of which are hereby incorporated by reference.

### FIELD OF THE INVENTION

The present invention relates generally to computer assisted orthopedic surgery, and more specifically to computer assisted placement of non-spherical implants that require a precise configuration.

### BACKGROUND OF THE INVENTION

In total hip arthroplasty, implants are used to replace the ball and socket joint of the hip to restore a subject's natural function. The joint is exposed and the femur and acetabulum are prepared, using reamers and broaches, to receive the implants. The femoral implant generally consists of a stem, neck and ball portion while the acetabular implant generally consists of an outer and inner shell. FIG. 1 is a prior art perspective view of a modular type femoral implant 100 that is designed with the stem 105, neck 103 and head portion 101 as separate components. Manufacturers provide different sizes and shapes for each component of a modular type femoral implant so the components may be assembled by the surgeon in a configuration that best fits the subject. While there are many different types of hip implants, one universal design characteristic is the rotational symmetry of the modular head component 101 about the longitudinal implant neck axis 107.

However, research has shown that the true shape of a healthy human femoral head is an ellipsoid rather than a sphere. There have been many proposed clinical benefits to the elliptical shape including improved mechanical properties and cartilage health. For example, a group performed computer simulated studies on the mechanical behavior of elliptical shaped heads vs. spherical shaped heads. The study reported that the ellipsoid model behaves better than a sphere in terms of acetabular deformation and acetabular peak stresses under static conditions.

Similarly, the components of an acetabular implant are generally designed as perfect hemispheres. However, the acetabulum in fact has a morphologically undulating rim. The undulations consist of peaks and valleys where important muscles and tendons naturally align and follow. For example, the psoas valley on the anterior rim of the acetabulum provides an anatomical path for the iliopsoas tendon. It has been shown that protruding or mal-aligned acetabular components may cause iliopsoas impingement, which may lead to tendon irritation and tearing. As the manufacturing of asymmetric, non-spherical and/or implants with unique features are possible, the placement of the components within the subject requires a high degree of precision and accuracy.

Recently, with the advancements in three dimensional (3-D) printing, subject specific implants have been proposed as a possible alternative to traditional implants. 3D printed implants could take advantage of natural and healthy shaped anatomy that could provide a clinical benefit to the subject and to the longevity of the implant. However, there is still a need to optimally place the implants in the subject intra-operatively to ensure the orientation and any crucial landmarks of the implant are in the correct location precisely.

Thus there exists a need to provide the surgeon and subject with an implant that can take advantage of the natural shapes of the anatomy to provide a better clinical outcome. There further exists a need for the precise placement of the implant in the subject.

### SUMMARY OF THE INVENTION

A method for implantation of non-spherical, asymmetric implants is provided that includes devising a pre-surgical plan with pre-operative planning software operating on a computer to define at least one of shape, orientation, type, size, geometry, or placement of the non-spherical, asymmetric implant in an operative bone of a subject. A computer assisted surgical device is used to place the non-spherical, asymmetric implant. The implant is positioned within the bone by the computer assisted surgical device in accordance with pre-surgical plan.

A non-spherical, asymmetric implant for insertion in a bone is provided that includes a stem for insertion into a femur of a subject. A neck is connected to the stem. A head portion is provided that is adapted to fit into an acetabulum of the subject and is attached to the neck. The stem, the neck, and the head portion are separate components adapted to fit together.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts a typical modular hip implant of the prior art with a femoral head rotationally symmetric about the implant neck axis in accordance with embodiments of the invention;

FIG. 2 depicts an inventive modular femoral head implant design that has only two orders of rotational symmetry along three axes in accordance with embodiments of the invention;

FIG. 3 depicts a modular assembly of the inventive head design of the implant and the effect of rotating the component about the neck axis in accordance with embodiments of the invention;

FIG. 4 illustrates an acetabular cup implant with unique features in accordance with embodiments of the invention;

FIG. 5 depicts a modular neck rotating about the stem to an optimal orientation in accordance with embodiments of the invention; and

FIG. 6 illustrates a method to fix the femoral head on the neck in a preferred orientation in accordance with embodiments of the invention.

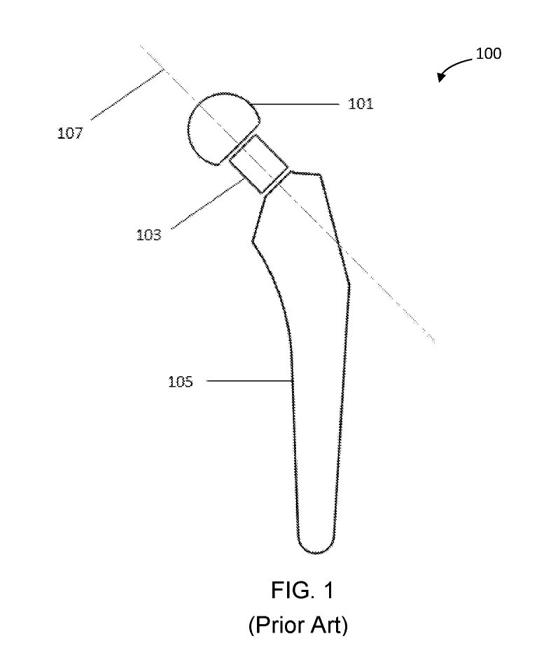

### DESCRIPTION OF THE INVENTION

The invention disclosed herein describes asymmetrical, non-spherical, and/or implants with unique features and methods for implantation, but more particularly to the planning and execution of joint replacement surgery with asymmetrical, non-spherical, and/or implants with unique features with computer assisted devices.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

The invention disclosed herein has utility for the implantation of non-spherical, asymmetric, and/or implants with unique features that provide a clinical benefit to the subject and/or the longevity of the implant. It should be appreciated that research suggests that naturally shaped implants could provide a clinical benefit especially if precisely placed in the proper location and orientation in the subject.

Reference will be made herein to the replacement of hip joints and knee joints and it should be understood that the present invention may be applied to other joints within the body and any other bones found within the body. These other joints that are repaired through resort to the present invention illustratively include the hip joint, shoulder joint, ankle joint, wrist joint, finger joint, toe joint, or other joint. As used herein, a subject is defined as a human; or an animal of a non-human primate, a horse, a cow, a sheep, a goat, a dog, a cat, a rodent and a bird; and a non-living cadaver of any of the aforementioned.

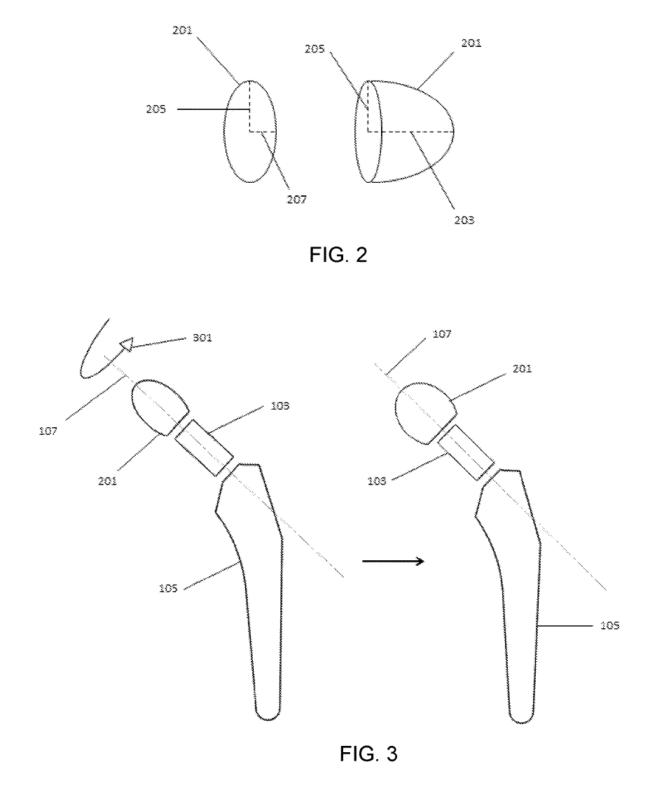

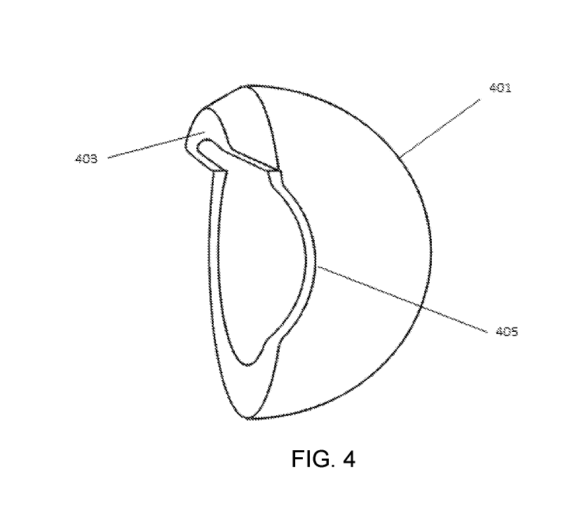

Referring now to the figures, FIG. 2 depicts a modular femoral head implant design 201 that has only two orders of rotational symmetry along three axes in accordance with embodiments of the invention. The femoral head implant 201 has different dimensions about all three axes 203, 205 and 207. In a specific embodiment, the implant head 201 is designed as an ellipsoid. The radius along axis 203 is greater than the radius along axis 205. The radius along axis 205 is greater than the radius along axis 207. In a specific embodiment, the rotational axis of the femoral head 201 aligned with neck axis 107 is axis 203. With respect to FIG. 3, when the head 201 is rotated (represented by arrow 301) about the neck axis 107, the geometric relationship of the femoral head 201 changes with respect to the neck 103 and stem 105. In traditional total hip arthroplasty, manually placing the femoral head 201 on the neck 103 in the correct orientation to best match a pre-surgical plan or the true shape of the subject's anatomy would be difficult. However, this limitation can be overcome with the use of a computer assisted surgical device.

In specific embodiments, any combination of the different radii between each axis can be accomplished whereby FIG. 2 simply illustrates one example of an implant with different dimensions about all three axes 203, 205, and 207. Other implant components can likewise be asymmetric, non-spherical, or have unique features that would provide a clinical benefit to the subject. For example, an acetabular component 401 with unique features is illustratively shown in FIG. 4. In one embodiment the acetabular component has an extruding portion 403 at the top of the implant. The extruding portion may allow for more stability, congruency, and load transfer for use with a traditional femoral head implant or a femoral head implant that is asymmetric, non-spherical, and/or has a unique feature. In another embodiment, the acetabular component can have a recess 405 at the rim that can provide additional space for the iliopsoas tendon as an example. Due to the unique features, manual implantation could prove difficult to get the unique features in the correct position and/or orientation. Therefore, a computer assisted device can be used to precisely place the implant so the unique features are in an optimal position to provide the best clinical benefit to the subject.

As shown above total hip arthroplasty is one implementation that benefits from embodiments the invention, and the use of asymmetric, non-spherical and/or implants with unique features can be advantageous for other applications as well. For example, the implants and computer assisted implantation may be used in other surgical contextual locations such as the knee joint, hip joint, spine, shoulder joint, elbow joint, ankle joint, jaw, tumor site, joints of the hand or foot, and other appropriate surgical sites. In a specific embodiment, a hip resurfacing implant may have an elliptical shape.

### Pre-Operative Implant Planning

In a specific inventive embodiment, pre-operative planning software may be used to determine the best shape, orientation, type, size, geometry, and placement, of an implant in the operative bone. The operative bone may be represented in the pre-operative software as two-dimensional images or three-dimensional virtual models as known in the art. The pre-operative planning software may have a database of pre-loaded manufacturer implants that the user may choose from to optimally plan the surgery. The manufacturer implants may include implants with non-spherical, asymmetric or unique features that have already gained regulatory approval. In another specific embodiment, generic virtual models of an implant may be chosen by the user, whereby the shape of the generic virtual model may be modified and then sent to a third party to be manufactured. For example, the generic virtual model of a femoral head may be a sphere, represented as a triangular mesh, whereby the user may adjust the diameters into an elliptical shape that the user deems is the most appropriate for the subject. In another embodiment, the pre-operative planning software creates a virtual model of the bone and automatically creates a subject specific implant according to the subject's anatomy. The shape or geometry of the subject specific implant may be created based on the natural and healthy shape of the bone in cases of bone deformity. In certain embodiments, the natural or healthy shape of the subject's contralateral side may be used to create the subject specific implant.

In a specific embodiment, the implant components are modular. The pre-operative planning software allows the user to select or design individual components of the overall implant. For example, the user may choose from a database of different stems, necks, and femoral heads that may be assembled virtually that would provide the best clinical outcome and/or implant survival rate. In one embodiment, the pre-operative planning software allows the user to choose from one or more manufactured modular components while allowing the customization of any of the remaining components. For example, the user may choose a regularly manufactured stem and neck while the femoral head is custom designed. In a specific embodiment, the pre-operative planning software can automatically ensure that the custom component is designed to precisely fit the regularly manufactured components. For example, if the femoral head is automatically designed by the pre-operative planning software or by the user then the software will ensure the head can be optimally assembled on the desired neck component.

In another specific embodiment, when an implant is non-spherical, asymmetric or has a unique feature, the pre-operative planning software may put constraints on, and/or automatically assist in the choice and/or design of the opposing component(s) to ensure optimal fit and performance. For example, the user may design or choose an elliptical femoral head first whereby the shape of the femoral head puts constraints on the design or reduces the number of choices for the acetabular component. Therefore, the software ensures all of the components of the procedure may be optimally and safely assembled within the subject and according to the user's pre-operative plan.

### Computer Assisted Implant Placement

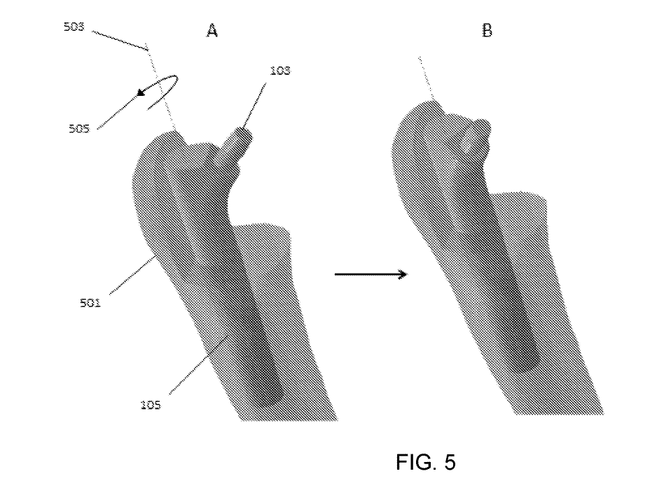

Intra-operatively, a computer assisted surgical device may assist a surgeon in preparing the bone and precisely placing the non-spherical, asymmetric and/or implant with a unique feature. Examples of computer-assisted surgical devices include a serial-chain manipulator system, a parallel robotic system, a haptically controlled robotic system or a hand-held robotic system, such as those described in U.S. Pat. Nos. 5,086,401, 7,206,626, and 8,961,536 all of which are hereby incorporated by reference in their entirety. In a specific inventive embodiment, the computer assisted surgical device precisely prepares the femoral canal according to the pre-operative plan so the desired alignment, fit, and fill of the stem component is achieved. In certain cases, the modular neck component may fit on the stem in different orientations. With respect to FIG. 5, a modular neck 103 is shown that may be fixed into place onto the stem 105 within the femur 501 at different orientations. In one embodiment, the computer assisted surgical device knows the position and orientation of the femur using known registration techniques such as point to surface registration as described in U.S. Pat. No. 6,033,415, which is hereby incorporated by reference in its entirety. The device also knows the orientation of the milled cavity from the cut instructions created during the pre-operative plan. The device may then optimally place the modular neck component 103 in the proper orientation with respect to the femur. For instance, as shown in FIG. 5, the device may rotate (shown by arrow 505) the modular neck 103 to the desired anteversion defined in the pre-surgical plan (from A to B) about axis 503. The neck 103 may then be fixed into place manually by the surgeon or by the surgical device using a fixation technique known in the art such as screws, press-fit, or pins. The device may also rotate recess 405 of the acetabular component 401 to obtain a desired acetabular anteversion or a combined femoral and cup anteversion.

In another specific embodiment, with respect to FIG. 2, the computer assisted surgical device can optimally orient a modular femoral head 201 on the neck 103. The device can attach to the head 201 using a technique known in the art such as a gripping clamp, a magnet, a reference hole on the implant that receives the end effector of the device. In one embodiment, a small receiving portion, such as a hole, is located in a specific location on the implant that provides the surgical device with the orientation of the implant relative to the end effector. In another embodiment, a digitizer can be used to collect points on the implant that can be used for registration with a virtual model of the implant to provide the device with the orientation of the implant. Once the initial orientation of the implant is known, the device can precisely orient the implant in the desired orientation. For example, the implants can be registered by digitizing a set of points on the implant so the device knows the initial orientation of the modular head 201 and the orientation of the neck 103, and subsequently rotate (shown by arrow 301) the head 201 to the desired orientation.

Component Fixation

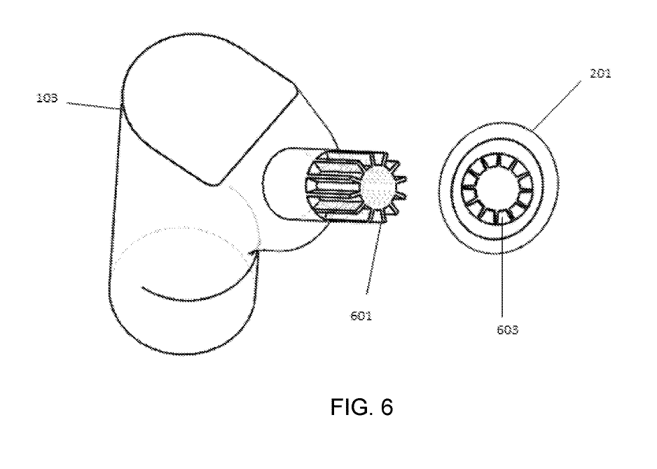

The asymmetrical, non-spherical, and/or implant with a unique feature may be designed to be fixed in the desired orientation relative to the other implant components and/or relative to the bone. In a specific embodiment, with respect to FIG. 6, the modular neck 103 that receives the femoral head component 201 may contain teeth and grooves 601 that allow the head 201 to be fixed when placed in the desired orientation on the neck 103. The portion of the femoral head 201 the fits over the neck 103 may contain corresponding teeth and grooves 603 that tightly fix the implant 201 into the desired orientation. Half the number of grooves would correspond to the number of different orientations the implant may be fixed for an implant with an axis of rotational symmetry having an order of 2. Depending on the number and design of the grooves, multiple orientations could exist that would require a surgical device to reference the neck 103 and the femoral head 201 in order to optimally place the femoral head 201. In a specific embodiment, when only a few orientations exist for the femoral head 201 to be placed on the neck 103, the user may directly place the head 201 and fix the head 201 into place. A device or pre-operative plan may be used to help assist the surgeon in placing the component head 201. For example, the teeth and grooves could be numbered whereby after surgical planning the surgeon knows that groove ‘x’ on the neck should fit with tooth ‘y’ of the head.

In another specific inventive embodiment, the implant may be fixed in the desired orientation using biocompatible reagents such as Poly(methyl methacrylate) (PMMA). The surgical robotic device, upon registering the implant, may optimally place the correct amount of the reagent at specific locations on the implant that are known to provide a sufficient fix. The surgical robotic device may then place the implant in the correct orientation. In specific embodiments, the implants can be made of materials whereby upon contact with a reagent causes a biocompatible reaction that fixates the two contacting surfaces. In a specific embodiment, during the pre-planning stage, once the location and orientation of all the components have been placed, the components may be selected, designed and/or tailored so that the connecting portions may be designed to fix only in the desired orientations.

### Other Embodiments

The present invention also includes a business method in which one or more aspects of the method of pre-surgical planning, implant design, implant placement/positioning are performed for financial remuneration. The subject receiving an implant or a third party insurer is invoiced for such services. Payment is then conveyed by electronic transaction or financial instrument to the provider of the method for services rendered and the implant.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A method for implantation of non-spherical, asymmetric implants comprising:
devising a pre-surgical plan with pre-operative planning software operating on a computer to define at least one of shape, orientation, type, size, geometry, or placement of the non-spherical, asymmetric implant in an operative bone of a subject, the subject being a living human, a cadaver, or a living animal;
using a computer assisted surgical device to place the non-spherical, asymmetric implant; and
positioning the non-spherical, asymmetric implant with the computer assisted surgical device in accordance with pre-surgical plan.

2. The method of claim 1 wherein the pre-operative planning software has a database of pre-loaded manufacturer implants that a user chooses from to optimally plan the implantation surgery.

3. The method of claim 1 wherein the pre-operative planning software has generic virtual models of a plurality of implants to be chosen by the user, whereby one or more shapes of the generic virtual model are modified and the modified generic model is sent to a third party to be manufactured.

4. The method of claim 1 wherein the pre-operative planning software both constrains and automatically assists a user's choice or design of an opposing component to ensure a desired fit and performance of the non-spherical, asymmetric implant.

5. The method of claim 1 wherein the computer assisted surgical device prepares at least a portion of the operative bone for the placement of the non-spherical, asymmetric implant.

6. The method of claim 1 wherein the computer assisted surgical device rotates the non-spherical, asymmetric implant to an anteversion defined in the pre-surgical plan.

7. The method of claim 1 further comprising a digitizer used to collect points on the implant that are used for registration with a virtual model of the implant to provide the computer assisted surgical device with the position and orientation of the non-spherical, asymmetric implant.

8. The method of claim 1 further comprising fixating the non-spherical, asymmetric implant in place and orientation using biocompatible reagents.

9. The method of claim 8 wherein the biocompatible reagent is poly(methyl methacrylate) (PMMA).

10. The method of claim 8 wherein the non-spherical, asymmetric implant is made of materials whereby upon contact with the reagent causes a biocompatible reaction that fixates the two contacting surfaces of the implant and operative bone.

11. The method of claim 1 wherein the non-spherical, asymmetric implant is configured for implantation in at least one of a knee joint, a hip joint, a spine, a shoulder joint, an elbow joint, an ankle joint, a jaw, a tumor site, and joints of the hand or foot.

12. The method of claim 1 wherein the non-spherical, asymmetric implant further comprises a stem for insertion into a femur of the subject, a neck connected to said stem, and a head portion adapted to an acetabulum of the subject and attached to the neck, where the stem, the neck, and the head portion are separate components.

13. The method of claim 1 wherein at least three of the shape, the orientation, the type, the size, the geometry, and the placement of the non-spherical, asymmetric implant in an operative bone of a subject are determined by the pre-surgical plan.

14. The method of claim 1 wherein all of the shape, the orientation, the type, the size, the geometry, and the placement of the non-spherical, asymmetric implant in an operative bone of a subject are determined by the pre-surgical plan.

15. A non-spherical, asymmetric implant for insertion in a bone by the method of claim 1 comprising:
a stem for insertion into a femur of a subject;
a neck connected to said stem; and
a head portion adapted to an acetabulum of the subject and attached to the neck, where the stem, the neck, and the head portion are separate components.

16. (canceled)

17. The implant of claim 15 wherein the head portion is designed as an ellipsoid having three radii of three different dimensions.

18. A computer-assisted surgical system for assembling the implant of claim 15 in a planned position and orientation in the subject.
